(19) United States
(12) Patent Application Publication
(10) Pub. No.: US 2025/0154186 A1
(43) Pub. Date: May 15, 2025

(54) HYDROCARBON OXIDATION WITH DIOXYGEN IN IRON-CONTAINING METAL-ORGANIC FRAMEWORKS

(71) Applicant: THE REGENTS OF THE UNIVERSITY OF CALIFORNIA, Oakland, CA (US)

(72) Inventors: Jeffrey R. Long, Lafayette, CA (US); Jonas Boergel, Berkeley, CA (US); Kaipeng Hou, Richmond, CA (US)

(73) Assignee: THE REGENTS OF THE UNIVERSITY OF CALIFORNIA, Oakland, CA (US)

(21) Appl. No.: 19/009,009

(22) Filed: Jan. 3, 2025

Related U.S. Application Data
(63) Continuation of application No. PCT/US2023/070362, filed on Jul. 18, 2023.
(60) Provisional application No. 63/368,808, filed on Jul. 19, 2022.
(60) Provisional application No. 63/390,117, filed on Jul. 18, 2022.

(51) Int. Cl. C07F 15/02; B01J 31/16; C07C 45/33

(52) U.S. Cl. CPC C07F 15/025; B01J 31/1691; C07C 45/33; B01J 2231/70; B01J 2531/842; C07C 2601/14

(57) ABSTRACT

Metal organic framework FeₓZn₅₋ₓ(O₂CC(O)R)₄(btdd)₃ for hydrocarbon oxygenation where R is an alkyl or aryl substituent compositions as illustrated with FeₓZn₅₋ₓ(prv)₄(btdd)₃ where (x=1.0 to 1.8) and FeZn₄(moba)₄(btdd)₃, are provided. High-spin Fe(IV)=O species are generated using O₂ in a synthetic system that mimics α-ketoglutarate-dependent dioxygenases. Dioxygen activation yields a synthetic material capable of hydrocarbon oxidation catalysis at ambient temperatures via a high-spin iron(IV)-oxo intermediate. In the presence of O₂, the frameworks are capable of catalytic oxygenation of cyclohexane and the stoichiometric conversion of ethane to ethanol and methane to methanol at ambient temperatures, for example. The frameworks allow for the catalytic oxygenation of a variety of hydrocarbons and conversions to alcohols.

### CROSS-REFERENCE TO RELATED APPLICATIONS

This application claims priority to, and is a 35 U.S.C. § 111(a) continuation of, PCT international application number PCT/US2023/070362 filed on Jul. 18, 2023, incorporated herein by reference in its entirety, which claims priority to, and the benefit of, U.S. provisional patent application Ser. No. 63/390,117 filed on Jul. 18, 2022, incorporated herein by reference in its entirety, and which also claims priority to, and the benefit of, U.S. provisional patent application Ser. No. 63/368,808 filed on Jul. 19, 2022, incorporated herein by reference in its entirety. Priority is claimed to each of the foregoing applications.

The above-referenced PCT international application was published as PCT International Publication No. WO 2024/020357 A1 on Jan. 25, 2024, which publication is incorporated herein by reference in its entirety.

### STATEMENT REGARDING FEDERALLY SPONSORED RESEARCH OR DEVELOPMENT

This invention was made with Government support under Grant Number DE-SC0019992, awarded by the U.S. Department of Energy. The Government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to catalytically active metal organic framework (MOF) systems and methods, and more particularly to compositions, systems and methods for the production of reactive high-spin Fe(IV)=O intermediates generated directly from dioxygen. The described frameworks are also capable of performing a variety of stoichiometric and catalytic hydrocarbon oxygenation reactions.

### 2. Background

The development of catalysts for the selective oxygenation of light hydrocarbons using O₂ remains a formidable but important challenge in the global effort to reduce dependence on crude oil. To mitigate the consequences of human-made climate change, improved chemical processes with significantly less energy demand and greenhouse gas emissions need to be developed. Natural gas consists primarily of methane, which is a more potent greenhouse gas than carbon dioxide and difficult to store. The efficient conversion of methane to methanol with dioxygen as the stoichiometric oxidant would allow one to facilitate the storage because methanol is a liquid at ambient conditions and to prevent the uncontrolled release of methane into the atmosphere. Furthermore, methanol can be used as a commodity feedstock for fine chemical production, decreasing the reliance on crude oil. However, reports of suitable materials for the energy-efficient partial oxygenation of methane to methanol are scarce because of the chemical challenges associated with the requirements of using abundant dioxygen and close to ambient reaction temperatures.

Nature has developed mononuclear nonheme iron metalloenzymes that utilize O₂ for C—H oxygenation chemistry, such as the ubiquitous α-ketoglutarate-dependent dioxygenases. One well-studied enzyme in this class is taurine-α-ketoglutarate dioxygenase (TauD), which oxygenates one of the C—H bonds of taurine alpha to the sulfonate group. Key to the reactivity of TauD and its family of dioxygenases is a high-spin (S=2) Fe(IV)=O intermediate, which is formed following oxidation of iron(II) with O₂ coupled with oxidation and decarboxylation of the α-ketoglutarate co-substrate (FIG. 2A). Over the last several decades, significant research effort has been devoted to the design and study of iron(IV)-oxo species in molecular and iron-zeolite model systems in order to better understand and mimic their reactivity in biological systems. However, the majority of examples studied to date are low-spin, and only a small number of these are generated using dioxygen in solution. High-spin Fe(IV)=O species have been accessed with oxidants such as trimethylammonium-N-oxide, hypervalent iodine reagents, and nitrous oxide, but the use of O₂, akin to metalloenzyme reactivity, for the generation of a high-spin Fe(IV)=O species has yet to be achieved in any synthetic system.

Metal-organic frameworks have received increasing attention in recent years as attractive systems for studying biomimetic chemistry. These porous, crystalline solids are constructed from metal nodes and organic linkers, and they exhibit chemical and structural tunability that is unmatched in other porous materials. As such, metal-organic frameworks offer the opportunity to explore O₂ activation in solid-gas reactions, while the immobilization of metal sites in the lattice may serve to prevent the decomposition of reactive species via dimerization or intramolecular ligand oxidation pathways available to molecular compounds. However, reported mimics of nonheme iron enzymes in metal-organic frameworks are scarce.

Therefore, there is a need for an enzyme-inspired solid catalyst material that will address said challenges.

### BRIEF SUMMARY

In nature, nonheme iron-containing enzymes use dioxygen to generate high-spin Fe(IV)=O species that is capable of participating in a variety of oxygenation reactions. Although scientists have long sought to mimic this reactivity, the use of O₂ to form high-spin Fe(IV)=O species that is akin to metalloenzyme reactivity, remains an unrealized goal in synthetic chemistry. The present technology provides a metal-organic framework featuring iron(II) sites with a local structure reminiscent of that in α-ketoglutarate-dependent dioxygenases. Specifically, the iron(II) sites in the frameworks FeₓZn₅₋ₓ(prv)₄(btdd)₃ and FeZn₄(moba)₄(btdd)₃ activate O₂ at 100 K to form reactive high-spin Fe(IV)=O species.

The preferred frameworks, FeₓZn₅₋ₓ(prv)₄(btdd)₃ (x=1 or 1.8; Hprv=pyruvic acid) and FeZn₄(moba)₄(btdd)₃ (Hmoba=3,3-dimethyl-2-oxobutanoic acid), are prepared from FeₓZn₅₋ₓCl₄(btdd)₃ via a post-synthetic ligand exchange of chloride for the corresponding α-ketocarboxylate (FIG. 2B and FIG. 2C). Although the frameworks with PRV and MOBA substituents are used to illustrate the class, other alkyl or aryl substituents (R) can be used as shown in the structures of FIG. 10. This framework material is generally denoted as FeₓZn₅₋ₓ(O₂CC(O)R)₄(btdd)₃, where R is an alkyl or aryl substituent. Preferred alkyl or aryl substituents include Et, n-Bu, t-Bu, 1-Pr, CF₃, Ph, Tol, C₆H₄CF₃ and C₆H₄OMe.

Exposure of FeₓZn₅₋ₓ(prv)₄(btdd)₃ to dioxygen at 100 K results in the direct formation of spectroscopically observable high-spin Fe(IV)=O species, reactivity that is without precedent in a synthetic system. The Fe(IV)=O sites have been characterized using in situ diffuse reflectance infrared Fourier transform spectroscopy (DRIFTS), in situ Mössbauer spectroscopy, and nuclear resonance vibrational spectroscopy. An S=2 spin ground state is assigned for the Fe(IV)=O species based on analysis of variable magnetic field Mössbauer and Fe Kβ x-ray emission spectra collected for O₂-dosed FeZn₄(moba)₄(btdd)₃. Finally, in the presence of O₂, the FeₓZn₅₋ₓ(prv)₄(btdd)₃ framework is competent for the catalytic oxygenation of cyclohexane and for the activation of even stronger C—H bonds, as demonstrated by the oxidation of ethane to ethanol and methane to methanol at low temperatures.

Significantly, the frameworks described herein are also a rare non-enzymatic system capable of catalytic hydrocarbon oxygenation and the first system that can oxidize ethane to ethanol and methane to methanol at ambient temperatures via a reactive high-spin Fe(IV)=O intermediate generated directly from dioxygen. In the presence of O₂, the frameworks are highly reactive for the catalytic oxygenation of cyclohexane.

According to one aspect of the technology, an iron-containing metal-organic framework (MOF) is presented that comprises FeₓZn₅₋ₓ(prv)₄(btdd)₃ where x=1.0 to 1.8 or FeZn₄(moba)₄(btdd)₃. By way of example, and not of limitation, these frameworks can be used for chemoselective oxygenation of hydrocarbons by exposing the MOF to oxygen gas and at least one hydrocarbon substrate, and then collecting resultant reaction products.

According to another aspect of the technology, a MOF bed comprising FeₓZn₅₋ₓ(prv)₄(btdd)₃ can be used for catalytic oxidation of cyclohexane to cyclohexanol and cyclohexanone in the presence of excess α-ketoacid, under an atmosphere of oxygen gas. In one embodiment the α-ketoacid comprises pyruvic acid.

A further aspect of the technology is to use a MOF bed comprising FeₓZn₅₋ₓ(prv)₄(btdd)₃ that can be used for stoichiometric oxidation of ethane to ethanol and acetaldehyde by treatment of the MOF with a mixture of oxygen gas and ethane and then collecting resultant ethanol and acetaldehyde or oxidation products. This MOF bed can also be used for oxidation of methane to methanol by treatment of the MOF with a mixture of oxygen gas and methane and then collecting resultant methanol oxidation products.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1 is a functional block diagram of a method for catalytic oxidation of a hydrocarbon according to one embodiment of the technology.

FIG. 2A is an illustration of the local structure of the mononuclear nonheme iron(II) sites in TauD and generation of the reactive high-spin iron(IV)=O species (TauD-J via oxidation with O₂ coupled with decarboxylation of the α-ketoglutarate co-substrate.

FIG. 2B is an illustration of the local coordination environment of the iron(II) sites in FeZn₄(prv)₄(btdd)₃ and FeZn₄(moba)₄(btdd)₃ that has observed reactivity with O₂ at low temperatures to form a high-spin iron(IV)=O species coordinated by acetate formed via the decarboxylation of pyruvate according to one embodiment of the technology.

FIG. 2C is a schematic illustration of the cubic pore of FeZn₄(prv)₄(btdd)₃ using single-crystal x-ray diffraction data obtained for Zn₅(prv)₄(btdd)₃ (left) and expanded view (right) of the truncated structure of a cluster node of the framework showing the nature of the pyruvate coordination, as supported by Mossbauer and nuclear resonance vibrational spectroscopies showing the design of a metal-organic framework mimic of taurine-α-ketoglutarate dioxygenase (Tau-D).

FIG. 3A is a plot of reactivity between FeₓZn₅₋ₓ(prv)₄(btdd)₃ and O₂ using variable-temperature in situ DRIFTS. DRIFTS spectra were obtained after dosing Fe_{1.8}Zn_{3.2}(prv)₄(btdd)₃ with 20 mbar O₂ at 100 K and gradually warming to 298 K (solid lines) and after dosing of Fe_{1.8}Zn_{3.2}(1-¹³C-prv)₄(btdd)₃ with 20 mbar ¹⁸O₂ at 100 K and gradually warming to 298 K (dashed lines).

FIG. 3B is a plot of spectra obtained using variable-temperature in situ DRIFTS showing the signature peaks for the iron(IV)=O species formed from the reaction between Fe_{1.8}Zn_{3.2}(prv)₄(btdd)₃ and O₂ (831 cm⁻¹) and Fe_{1.8}Zn_{3.2}(1-¹³C-prv)₄(btdd)₃ and ¹⁸O₂ (796 cm⁻¹). All data shown correspond to difference spectra obtained using the desolvated iron(II) frameworks as the background. Minor differences in the intensities of the absorption bands for the natural abundance and heavier isotopologue samples are likely due to differences in powder sample mass, sample distribution in the background matrix, and slight positioning differences in the infrared beam.

FIG. 3C is the reaction probed by DRIFTS, wherein FeZn₄(1-¹³C-prv)₄(btdd)₃ is shown to react with ¹⁸O₂.

FIG. 4 is a plot of in situ Mossbauer spectra collected at 5 K for desolvated FeZn₄(prv)₄(btdd)₃ before and after dosing with 300 mbar of O₂ at 100, 125, and 150 K. All spectra were fit with a minimum number of symmetric quadrupole doublets, all of which have the same linewidth for a given spectrum. The sub-spectra, respectively, are consistent with iron(II), iron(III), and iron(IV) species.

FIG. 5 is a plot of variable magnetic field Mossbauer spectra collected at 1.7 K and the indicated fields for a sample of FeZn₄(moba)₄(btdd)₃ that had been dosed with 100 mbar of O₂ at 100 K, held for 2 hours at 200 K and subsequently dosed with 200 mbar of O₂ at 100 K and then warmed again at 200 K for 2 hours. Subspectra were modeled as S=2 iron(II), S= 5/2 iron(III), isolated S=2 Fe(IV)=O species, and S=0 species arising from antiferromagnetic coupling between Fe(IV)=O species within the same node. The spectra were modeled as described in the main text to extract zero-field splitting and hyperfine coupling parameters for the uncoupled Fe(IV)=O species.

FIG. 6A is a nuclear resonance vibrational spectroscopy (NRVS) plot of iron partial vibrational density of states (PVDOS) distribution of ⁵⁷Fe-enriched, desolvated FeZn₄(prv)₄(btdd)₃ from NRVS data collected at ˜100 K and DFT computed Fe PVDOS for FeZn₄(prv)₄(bta)₆ overlayed.

FIG. 6B is a plot of iron PVDOS distribution of ⁵⁷Fe-enriched, desolvated FeZn₄(prv)₄(btdd)₃ obtained from NRVS data collected at ˜100 K after dosing with 200 mbar of O₂ at 163 K and DFT computed PVDOS distribution for ˜70% FeZn₄(prv)₄(bta)₆ and ˜30% of Fe(O)(κ²—OAc)Zn₄(prv)₃(bta)₆ overlayed. The inset shows a shift of the Fe(IV)=O vibration to lower wavenumbers (Δ=34 cm⁻¹) when ¹⁸O₂ is used.

FIG. 6C are the assigned vibrational modes of FeZn₄(prv)₄(btdd)₃ and the O₂-dosed framework (V₀ through V₃ shown in FIG. 6A and FIG. 6B).

FIG. 7 is a catalytic oxidation of cyclohexane to cyclohexanol and cyclohexanone illustrating one reaction of the frameworks FeₓZn₅₋ₓ (prv)₄(btdd)₃ with hydrocarbons and O₂.

FIG. 8 is a reaction of stoichiometric oxidation of ethane to ethanol and acetaldehyde shown schematically.

FIG. 9 is a representation of the oxidation of methane to methanol reaction according to one embodiment of the technology.

FIG. 10 is a schematic representation of other congeners of FeₓZn₅₋ₓ (prv)₄(btdd)₃ and their potential reaction with O₂. Some examples for R substituents are given, which generally denotes alkyl or aryl substituents.

### DETAILED DESCRIPTION

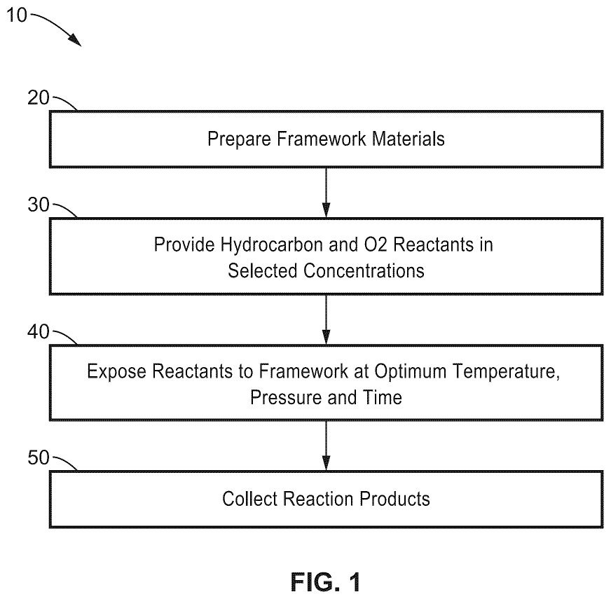

Referring more specifically to the drawings, for illustrative purposes, MOF compositions, systems and methods of fabrication and use are generally shown. Several embodiments of the technology are described generally in FIG. 1 to FIG. 10 to illustrate the characteristics and functionality of the compositions, systems, materials and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, a flow diagram of one method 10 for performing catalytic hydrocarbon oxygenation of a hydrocarbon source is shown schematically. The methods begin with the acquisition or fabrication of a catalytic framework at block 20.

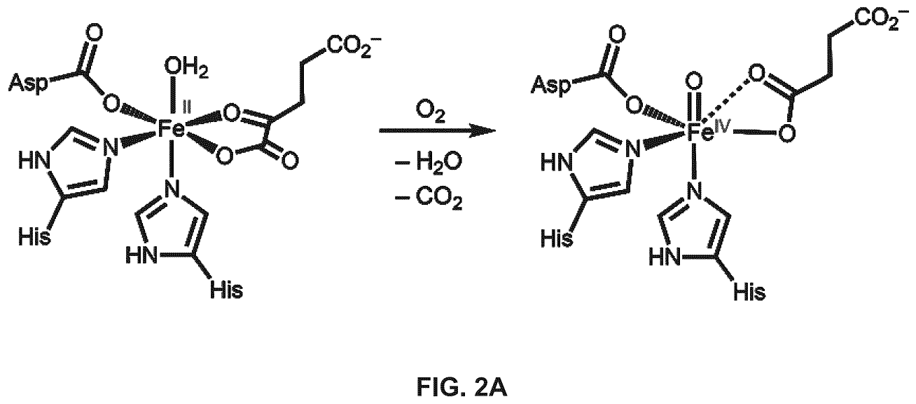

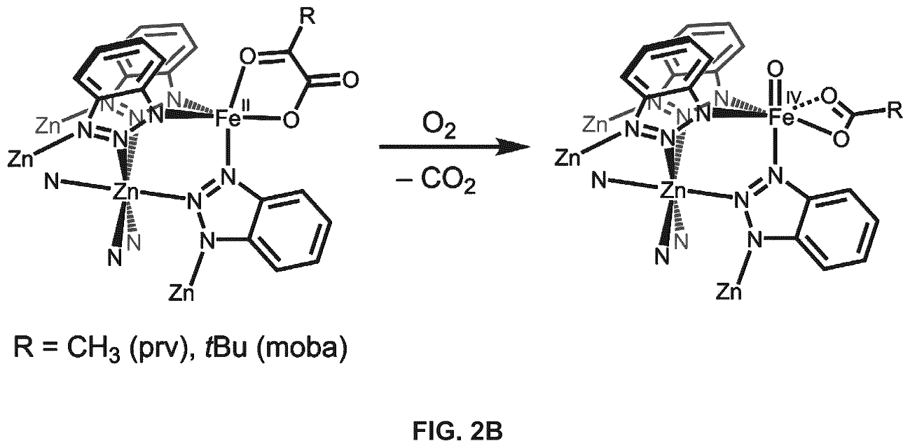

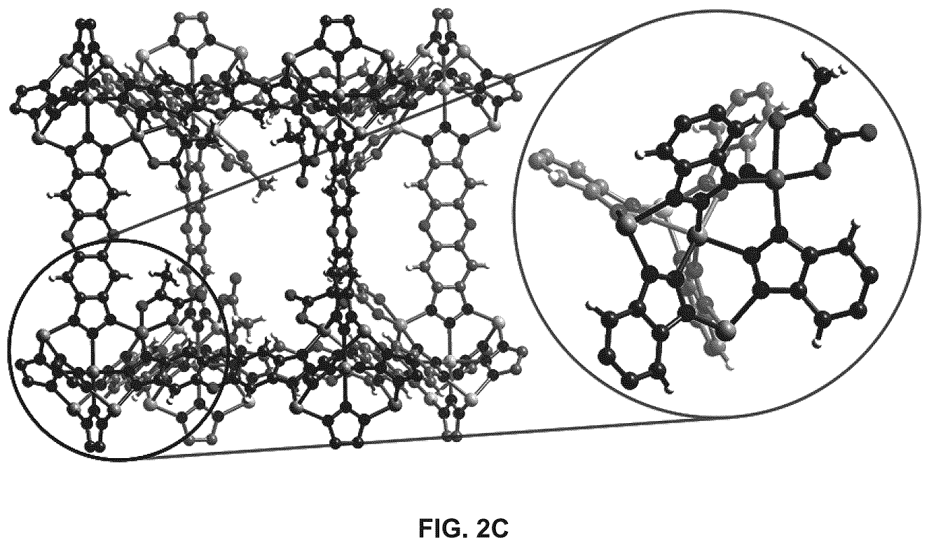

The preferred frameworks at block 20 are iron containing metal-organic frameworks of the type Zn₅Cl₄(btdd)₃ (MFU-4/), which consists of pentanuclear metal cluster nodes and bistriazolate ligands (H₂btdd=bis(1 H-1,2,3-triazolo [4,5-b],[4′,5′-i])dibenzo[1,4]dioxin) featuring cubic pores. The nodes feature a central zinc ion in octahedral geometry and four metal centers on the periphery of each cluster with a bound terminal chloride. The peripheral zinc ions can be partially exchanged for iron(II), and the terminally bound chloride can be replaced by pyruvate (prv) or other alkyl or aryl group containing derivatives, simple α-ketocarboxylates serving as additional two-electron reductants for the activation of dioxygen inspired by the mechanism of α-ketoglutarate dependent dioxygenases such as the taurine-α-ketoglutarate dioxygenase (TauD) as illustrated in (FIGS. 2A and 2B).

For the iron exchange, in one embodiment, Zn₅Cl₄(btdd)₃ (1 equiv) and FeCl₂ (4 equiv for x=1; 25 equiv for x=1.8) were suspended in N,N-dimethylformamide (DMF) at 50° C. for 20 hours, followed by removal of the supernatant and washes of the solid residue with DMF, methanol, acetonitrile, and dichloromethane to yield FeₓZn₅₋ₓCl₄(btdd)₃ (x=1.0-1.8).

For exchanging chloride with pyruvate, FeₓZn₅₋ₓCl₄(btdd)₃ (1 equiv) was soaked in a solution of pyruvic acid (100 equiv) and triethylamine (100 equiv) in acetonitrile at room temperature for 24 hours. The supernatant is removed, and the solid residue was washed with acetonitrile and dichloromethane followed by desolvation under dynamic vacuum at 120° C. for 6-12 h. Other synthesis methods are described in the Examples below.

Accordingly, the frameworks FeₓZn₅₋ₓ(prv)₄(btdd)₃ (x=1 or 1.8; Hprv=pyruvic acid) and FeZn₄(moba)₄(btdd)₃ (Hmoba=3,3-dimethyl-2-oxobutanoic acid), prepared from FeₓZn₅₋ₓCl₄(btdd)₃ via post-synthetic ligand exchange of chloride for the corresponding α-ketocarboxylate (FIGS. 2B and 2C). High-spin Fe(IV)=O species capable of hydrocarbon oxidation may be generated using O₂ in this synthetic system that mimics α-ketoglutarate-dependent dioxygenases.

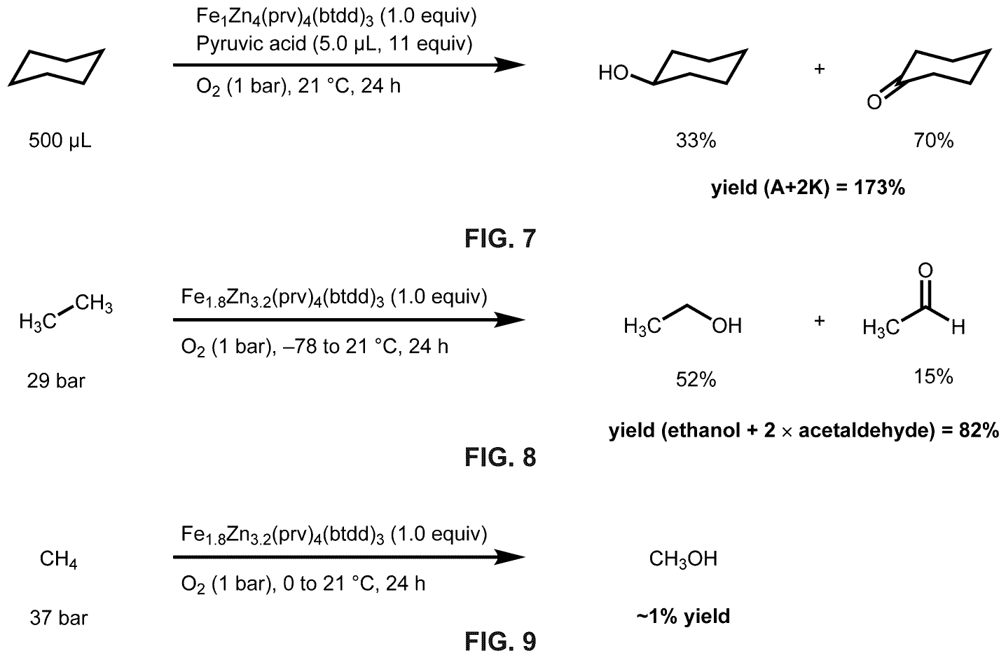

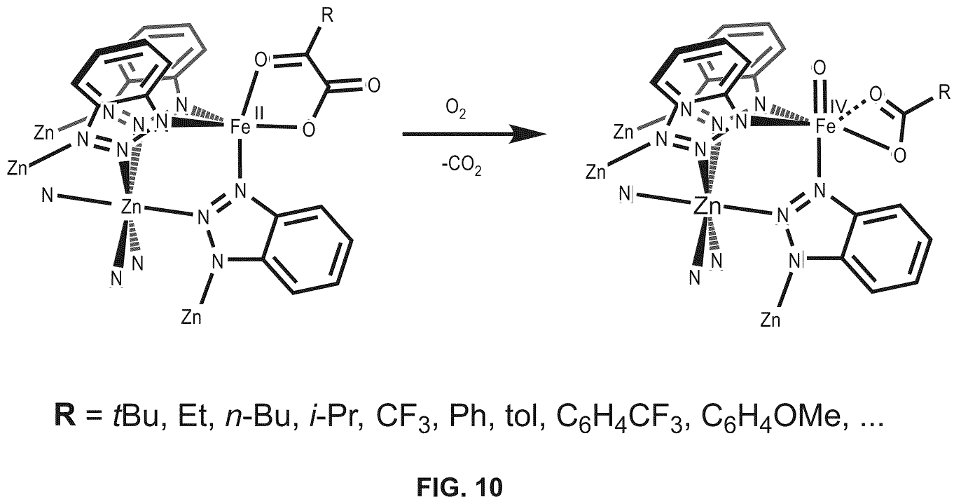

At block 30 the hydrocarbons are selected, and optimum oxygen temperature, pressure and reaction times may be determined to produce the desired products. The combination of dioxygen and hydrocarbon substrates lead to productive oxygenation of such to produce valuable alcohols directly at ambient temperatures with the potential for the use of such materials as catalysts, as demonstrated by the catalytic oxygenation of cyclohexane (FIG. 7). Moreover, ethane and even methanol were found to be competent substrates, which may enable the conversion of components of natural gas into value-added chemicals on industrially relevant scales (See e.g., FIG. 8, FIG. 9 and FIG. 10). Furthermore, the solid-gas reaction between the iron(II)-sites and O₂ at low temperatures enables their spectroscopic characterization, and thus allows the rational optimization of the Fe(IV)=O species to tune the reactivity toward hydrocarbons.

In situ diffuse-reflectance infrared Fourier transform spectroscopy, in situ Mössbauer spectroscopy, and nuclear resonance vibrational spectroscopy confirmed the identity of the reactive high-spin Fe(IV)=O species, corroborated by density functional theory calculations. Such reactive intermediates can form at temperatures as low as 100 K, however, in the absence of substrate their decomposition occurs above 200 K. Importantly, the framework stays crystalline and porous even after decomposition, further establishing its suitability for utilization in an industrial process.

It can be seen that the frameworks can be adapted with other formulations. Some examples for R substituents are given in FIG. 10, which generally denotes alkyl or aryl substituents. For example, R substituents can be one of Et, n-Bu, 1-Pr, CF₃, Ph, Tol, C₆H₄CF₃, C₆H₄OMe etc. The reaction conditions can be optimized to a particular hydrocarbon as well.

At block 40 of FIG. 1, the frameworks are exposed to the selected hydrocarbons and oxygen reactants. The materials are expected to show the best performance in a flow reactor setup. Methane or ethane and O₂ in a mixture of predominately hydrocarbon substrate can be flown over the system with a certain pressure depending on the setup. Vaporized pyruvic acid or other suitable volatile α-ketoacids can be continuously flown over the catalyst bed at a given temperature, mixed into the hydrocarbon and O₂ gas stream. The oxidation products can be collected continuously at an outlet at block 50. By-products from pyruvic acid decomposition, acetic acid and CO₂, possibly can be collected at the outlet as well. Alternatively, the material could be sequentially treated with the hydrocarbon-O₂ gas mixture and the α-ketoacid for the production of valuable alcohols.

It will be appreciated that metal-organic frameworks with open metal sites are especially well-suited for solid gas reactions because of their porosity and high density of sites. The technology described herein enables the chemoselective oxidation of methane to methanol and ethane to ethanol at low to ambient temperatures with dioxygen; factors that are important for an economically attractive process. The possibility of co-feeding pyruvic acid into a suitable reactor offers the potential for the development of an even more attractive catalytic process. Moreover, exploring the effects of various α-ketoacids (R=alkyl or aryl) is expected to have profound impact on the reactivity of the system because of changes in the electronic structure and altered steric properties, and thus the reactivity of the system, may be altered significantly (FIG. 10). Reported methodologies utilizing dioxygen suffer from the disadvantage that either high temperatures (>200° C.) are needed for dioxygen activation, such as in copper containing zeolites, or processes need regeneration of the active sites at elevated temperatures (200° C.) after sub-stoichiometric hydrocarbon oxidation in iron containing zeolites.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the structure and catalytic functionality of the frameworks, Fe_{X}Zn₅₋ₓCl₄(btdd)₃ (x=1 or 1.8) frameworks were synthesized and characterized. The frameworks were generally prepared using a post-synthetic cation exchange in Zn₅Cl₄(btdd)₃ using ferrous chloride. Energy-dispersive x-ray (EDX) spectroscopy revealed that the iron sites in FeₓZn₅₋ₓCl₄(btdd)₃ are homogeneously distributed within the materials and inductively coupled plasma optical emission spectroscopy (ICP-OES) confirmed the extent of iron substitution. Soaking FeₓZn₅₋ₓCl₄(btdd)₃ in an acetonitrile solution containing equimolar amounts of pyruvic acid and triethylamine resulted in essentially quantitative exchange of chloride for pyruvate and the formation of FeₓZn₅₋ₓ(prv)₄(btdd)₃.

For the synthesis of FeₓZn₅₋ₓCl₄(btdd)₃ (x=1 or 1.8), 20 mL N,N-dimethylformamide (DMF) was added to Zn₅Cl₄(btdd)₃ (MFU-4/) (0.120 g, 0.0951 mmol, 1.00 equiv) and FeCl₂ (50.0 mg, 0.395 mmol, 4.14 equiv for x=1) or FeCl₂ (0.300 g, 2.37 mmol, 24.9 equiv for x=1.8) in a 20 mL scintillation vial. The suspension was heated at 50° C. for 20 h. The supernatant was decanted, and the solid residue was soaked with 20 mL DMF for 12 hours. This process was repeated twice with DMF, six times with CH₃OH, three times with CH₃CN, and three times with CH₂Cl₂ and the total washing time was 180 h. The supernatant was removed, and the residue was heated under dynamic vacuum at 120° C. for 6-12 h to afford the desolvated frameworks FeZn₄Cl₄(btdd)₃ and Fe_{1.8}Zn_{3.2}Cl₄(btdd)₃ as yellow solids in quantitative yield.

To synthesize FeₓZn₅₋ₓ(prv)₄(btdd)₃ (x=1 or 1.8), a sample of FeZn₄Cl₄(btdd)₃ (0.120 g, 0.0959 mmol, 1.00 equiv) or Fe_{1.8}Zn_{3.2}Cl₄(btdd)₃ (0.120 g, 0.0964 mmol, 1.00 equiv) was suspended in CH₂Cl₂ (2 mL) in a 20 mL scintillation vial. To another 20 mL scintillation vial, pyruvic acid (846 mg, 9.61 mmol, 100 equiv) was added into CH₃CN (18 mL) and triethylamine (1.34 mL, 973 mg, 9.61 mmol, 100 equiv) was added. The mixture was then transferred to the 20 mL vial containing the framework suspension. After 24 hours, the supernatant was decanted, and the solid residue was soaked with 20 mL CH₃CN. This process was repeated four times with CH₃CN and three times with CH₂Cl₂ and the total washing time was 36 h. The supernatant was removed, and the residue was heated under dynamic vacuum at 120° C. for 6-12 h to afford the desolvated framework FeZn₄(prv)₄(btdd)₃ (0.110 g, 79%) or Fe_{1.8}Zn_{3.2}(prv)₄(btdd)₃ (0.105 g, 75%) as light-yellow powders. Analysis for C₄₈H₂₄N₁₈O₁₈FeZn₄: calculated: C, 39.54; H, 1.66; N, 17.29; found: C, 41.04; H, 2.23; N, 17.05. Analysis for C₄₈H₂₄N₁₈O₁₈Fe_{1.8}Zn_{3.2}: calculated: C, 39.75; H, 1.67; N, 17.38; found: C, 39.46; H, 2.22; N, 16.62.

Powder x-ray diffraction analysis confirmed that the FeₓZn₅₋ₓ(prv)₄(btdd)₃ materials are crystalline solids and isostructural to the parent MFU-4/framework, and N₂ adsorption data obtained at 77 K revealed high Brunauer-Emmett-Teller (BET) surface areas of 2130±12 and 2090±15 m²/g for x=1 and 1.8, respectively.

Analysis of Zn₅(prv)₄(btdd)₃ via single-crystal x-ray diffraction revealed that pyruvate coordinates to the peripheral zinc(II) centers in a bidentate fashion via the ketone and one of the carboxylate oxygen atoms. It was not possible to isolate single crystals of FeZn₄(prv)₄(btdd)₃ via single-crystal-to-single-crystal exchange starting from Zn₅(prv)₄(btdd)₃. However, the powder x-ray diffraction patterns of FeZn₄(prv)₄(btdd)₃ and Fe_{1.8}Zn_{3.2}(prv)₄(btdd)₃ are consistent with the simulated pattern generated for Zn₅(prv)₄(btdd)₃ from the single-crystal structure, which may indicate that the coordination mode of the pyruvate ligand is similar in the three frameworks.

Microcrystalline FeₓZn₅₋ₓ(prv)₄(btdd)₃ (x=1.0 or 1.8) and FeZn₄(moba)₄(btdd)₃ were loaded into 4 mm EPR tubes capped with J-Young adapters. Continuous-wave X-band EPR spectra were collected on the Bruker Biospin EleXsys E500 spectrometer with a dual mode cavity (ER4116DM) in parallel mode. Continuous-wave parallel mode electron paramagnetic resonance (EPR) spectroscopy and dc magnetic susceptibility data collected for FeₓZn₅₋ₓ(prv)₄(btdd)₃ further support the assignment of S=2 for the iron(II) sites.

### Example 2

A second framework from the group was prepared and tested to demonstrate the adaptability of the technology. To synthesize the framework of FeZn₄(moba)₄(btdd)₃, a sample of FeZn₄Cl₄(btdd)₃ (0.120 g, 0.0959 mmol, 1.00 equivalents was suspended in CH₂Cl₂ (2 mL) in a 20 mL scintillation vial. To another 20 mL scintillation vial, 3,3-dimethyl-2-oxobutanoic acid (1.25 g, 9.61 mmol, 100 equivalents) was dissolved in CH₃CN (18 mL) and triethylamine (1.34 mL, 973 mg, 9.61 mmol, 100 equivalents) was added. The mixture was then transferred to the 20 mL vial containing the framework suspension.

After 24 hours, the supernatant was decanted, and the solid residue was soaked with 20 mL CH₃CN. This process was repeated four times with CH₃CN and three times with CH₂Cl₂ and the total washing time was 36 hours. The supernatant was removed, and the residue was heated under dynamic vacuum at 120° C. for 6-12 hours to afford the desolvated framework FeZn₄(moba)₄(btdd)₃ (0.127 g, 81%) as a tan powder. Analysis for C₆₀H₄₈N₁₈O₁₈FeZn₄: calculated: C, 44.31; H, 2.97; N, 15.50; found: C, 43.21; H, 2.84; N, 15.75.

The local coordination environment of the iron(II) sites in FeZn₄(prv)₄(btdd)₃ (R=CH₃) or FeZn₄(moba)₄(btdd)₃ (R=tBu) were observed and the reactivity with O₂ at low temperatures to form an Fe(IV)=O species coordinated by acetate or pivalate formed via the decarboxylation of pyruvate (prv) or 3,3-dimethyl-2-oxobutyrate (moba), respectively is shown in FIG. 2B.

The synthesized FeZn₄(moba)₄(btdd)₃, which features a 3,3-dimethyl-2-oxobutyrate ligand with a tert-butyl group alpha to the carbonyl, was shown to be isostructural to FeₓZn₅₋ₓ(prv)₄(btdd)₃) and exhibits a comparably high BET surface area.

### Example 3

Reactivity between FeₓZn₅₋ₓ(prv)₄(btdd)₃ and O₂ was initially examined with variable-temperature in situ diffuse reflectance infrared Fourier transform spectroscopy (DRIFTS). For this purpose, the Fe_{1.8}Zn_{3.2}(prv)₄(btdd)₃ framework was used with the goal of maximizing the resulting spectral signal, while FeZn₄(prv)₄(btdd)₃ was used for the remaining spectroscopic analyses.

Following dosing of a sample of desolvated Fe_{1.8}Zn_{3.2}(prv)₄(btdd)₃ with 20 mbar of O₂ at 100 K, a new absorption band gradually appeared in the DRIFTS spectrum at 2341 cm⁻¹, which was assigned as the asymmetric C═O stretch of physisorbed CO₂ formed from decarboxylation of pyruvate as shown in FIG. 2A as solid lines. The intensity of this band increased as the temperature increased to 150 K and 200 K. Above 250 K, the band disappeared which was consistent with CO₂ desorption from the framework.

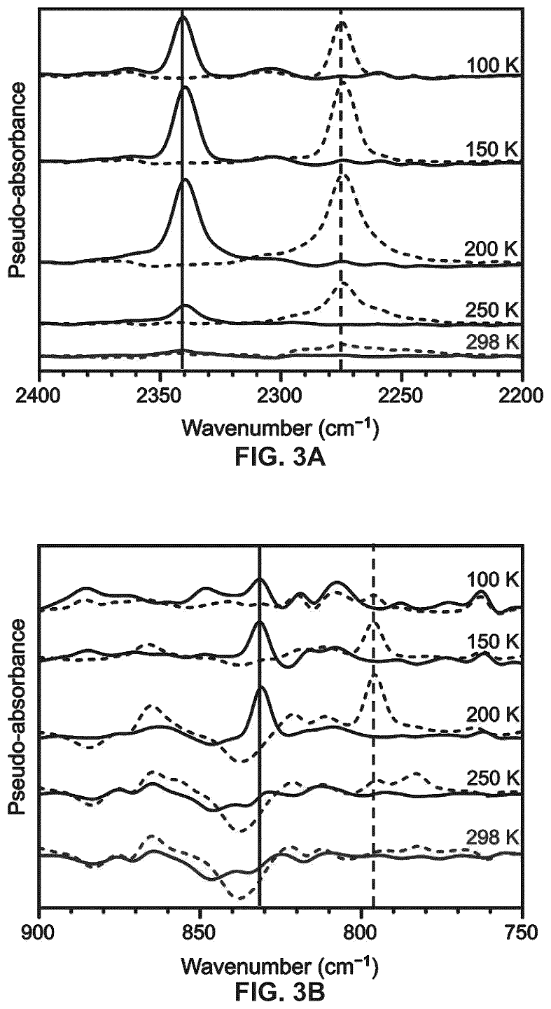

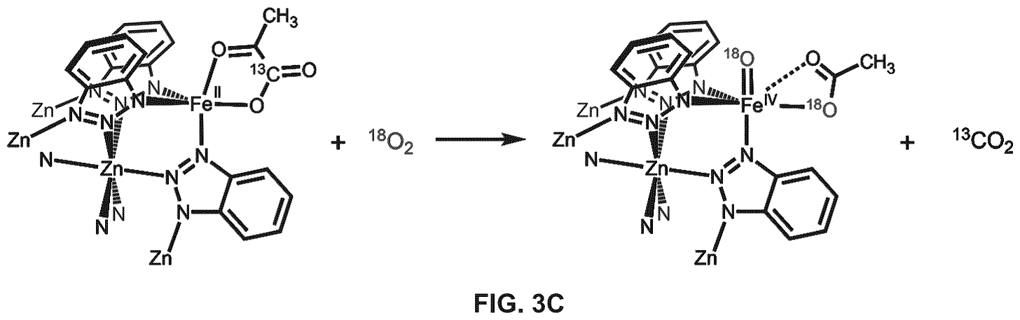

To verify that the detected CO₂ derived from pyruvate and not from O₂, an analogous in situ experiment was performed using Fe_{1.8}Zn_{3.2}(1-¹³C-prv)₄(btdd)₃ (1-¹³C-prv⁻=pyruvate labeled with ¹³C on the carboxylate carbon atom) and ¹⁸O₂. This is shown in FIG. 3A as dashed lines. Upon dosing with 20 mbar of ¹⁸O₂ at 100 K, a new stretch appeared at 2275 cm⁻¹, consistent with formation of the isotopologue ¹³CO₂ and not ¹³C¹⁸O₂, confirming that the oxygen atoms do not originate from dioxygen. As observed when using unlabeled pyruvate and O₂, the ¹³CO₂ stretch grew in intensity with heating to 200 K and disappeared at higher temperatures. A slight deviation of the experimental CO₂ stretching frequencies reported herein from the values associated with gas-phase CO₂ (2349 cm⁻¹ and 2284 cm⁻¹ for CO₂ and ¹³CO₂, respectively) can be ascribed to adsorption of the CO₂ within the pores of the framework at low temperatures.

Importantly, in situ powder x-ray diffraction data collected for FeZn₄(prv)₄(btdd)₃ after dosing with approximately 80 mbar of O₂ at 100 K over the course of gradual warming to 298 K revealed that the material remains highly crystalline under these conditions. In situ O₂ dosing DRIFTS experiments are shown in FIG. 3A and FIG. 3B. For the standard reaction (solid lines), desolvated FeZn₄(prv)₄(btdd)₃ was dosed with 20 mbar O₂ at 100 K and slowly heated to 298 K. For the isotopic labelling experiment (dotted lines), the framework sample was labeled with ¹³C at the carboxylic acid carbon of pyruvate and ¹⁸O₂ was used for dosing. The two peaks observed in the data obtained from the labelling experiment at higher temperatures suggest there may be scrambling of the oxygen atom from the proposed Fe(III)—¹⁸OH and ¹⁶O-atom of the formed acetate ligand.

In situ DRIFTS data collected for FeZn₄(moba)₄(btdd)₃ upon dosing with 20 mbar of O₂ at 100 K support Fe(IV)=O formation via decarboxylation of 3,3-dimethyl-2-oxobutyrate; v(Fe═O)=828 cm⁻¹, v(Fe=¹⁸O)=794 cm⁻¹), was consistent with data obtained for FeZn₄(prv)₄(btdd)₃ (v(Fe═O)=831 cm⁻¹, v(Fe=¹⁸O)=796 cm⁻¹).

An absorption band was also observed to grow in at 831 cm⁻¹ upon dosing Fe_{1.8}Zn_{3.2}(prv)₄(btdd)₃ with O₂ at 100 K, which was assigned as an Fe(IV)=O stretch and shown in FIG. 3B (solid lines). This band increased in intensity with heating up to 200 K before diminishing significantly at 250 K and disappearing at 298 K. When the analogous experiment was performed with ¹⁸O₂, the band shifted to 796 cm⁻¹, consistent with a stretching frequency of 794 cm⁻¹ calculated for Fe(IV)=¹⁸O using a simple harmonic oscillator model (FIG. 3B, dashed lines). For comparison, the Fe(IV)=¹⁶O and Fe(IV)=¹⁸O stretches in TauD-Jappear at 821 and 787 cm⁻¹, respectively. Concomitant with the disappearance of the Fe(IV)=O stretch at 250 K, a new stretch appeared at 3628 cm⁻¹. This stretch was attributed to an Fe(III)—OH species arising from decomposition of the Fe(IV)=O through hydrogen-atom abstraction, possibly from the methyl group of the newly formed acetate ligand (FIG. 2C). At 298 K, a new stretch was apparent at 3678 cm⁻¹, which may correspond to a different coordination environment for the Fe(III)—OH species at higher temperatures. When ¹⁸O₂ was used for dosing, a stretch appeared at 3617 cm⁻¹, in excellent agreement with that calculated for Fe(III)—¹⁸OH using a simple harmonic oscillator model (3616 cm⁻¹).

### Example 4

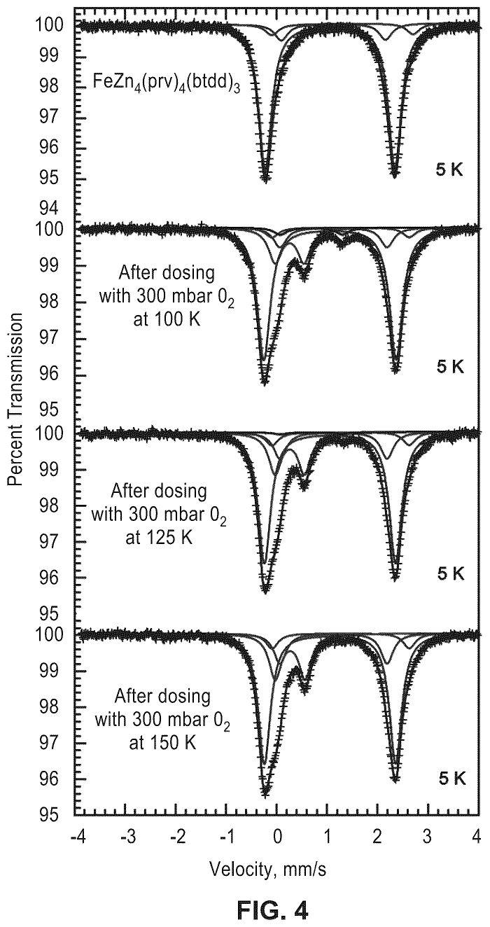

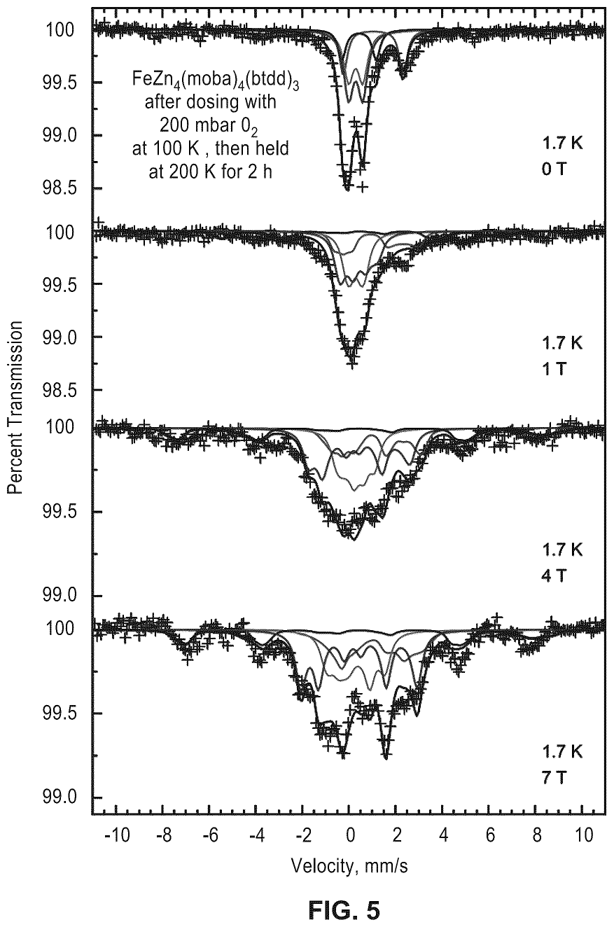

The species formed upon reaction of FeₓZn₅₋ₓ(prv)₄(btdd)₃ with dioxygen were further investigated by in situ Mossbauer spectroscopy. In situ Mossbauer spectra collected at 5 K for desolvated FeZn₄(prv)₄(btdd)₃ before and after dosing with 300 mbar of O₂ at 100, 125, and 150 K is shown in FIG. 4. Variable magnetic field Mossbauer spectra collected at 1.7 K and the indicated fields for a sample of FeZn₄(moba)₄(btdd)₃ that had been dosed with 100 mbar of O₂ at 100 K, held for 2 h at 200 K and subsequently dosed with 200 mbar of O₂ at 100 K and then warmed again at 200 K for 2 hours is shown in FIG. 5.

The zero-field Mossbauer spectrum of FeZn₄(moba)₄(btdd)₃ collected at 5 K featured a major doublet (86(1)% area; 6=1.059(1) mm/s and ΔE_{Q}=2.586(1) mm/s) was assigned to high-spin, five-coordinate iron(II). Following in situ dosing with 100 mbar O₂ at 100 K and heating at 200 K for 2 hours, a new quadrupole doublet is apparent in the 5 K Mossbauer spectrum with 6=0.292(1) mm/s and ΔE_{Q}=−0.603(1) mm/s (59.0(5)% area), assigned to the Fe(IV)=O species. When the sample was likewise dosed with a higher pressure of O₂ (200 mbar), the relative area of this doublet increased to 61.7(1)%. This relative area was significantly larger than the maximum relative area achieved upon dosing FeZn₄(prv)₄(btdd)₃ in situ with O₂ (21.1(1)%), supporting our hypothesis that intramolecular ligand oxygenation may be limiting the Fe(IV)=O content in FeZn₄(prv)₄(btdd)₃ following O₂ dosing.

Variable-field Mossbauer spectra were subsequently collected for O₂-dosed FeZn₄(moba)₄(btdd)₃ at 1.7 K under fields of 0, 1, 4, and 7 T and variable temperatures of 40, 15, 5, and 1.7 K under a field of 7 T is shown in FIG. 5. Spectra were also collected for FeZn₄(moba)₄(btdd)₃ at temperatures <5 K and fields of 0, 1, 4, and 7 T to obtain fixed parameters for modeling the iron(II) species in the variable-field Mossbauer spectra for the O₂-dosed material.

Consistent with the zero-field Mossbauer spectrum collected at 5 K, the zero-field spectrum for O₂-dosed FeZn₄(moba)₄(btdd)₃ collected at 1.7 K could be adequately fit with three subspectra, corresponding to an S=2 iron(II) component, an iron(Ill) species consistent with S= 5/2 (δ=0.440(4) mm/s, ΔE_{Q}=1.47(4) mm/s), and an Fe(IV)=O component (6=0.300(3) mm/s and ΔE_{Q}=−0.610(18) mm/s). The isomer shift of this Fe(IV)=O species is consistent with reported S=2 iron(IV)-oxo species in the literature.

### Example 5

Nuclear resonance vibrational spectroscopy (NRVS) was used to gain further insight into the local structure of the Fe(IV)=O species. This technique selectively yields the complete set of vibrational modes of Mossbauer-active nuclei and can therefore provide structural insights not accessible using other spectroscopic methods.

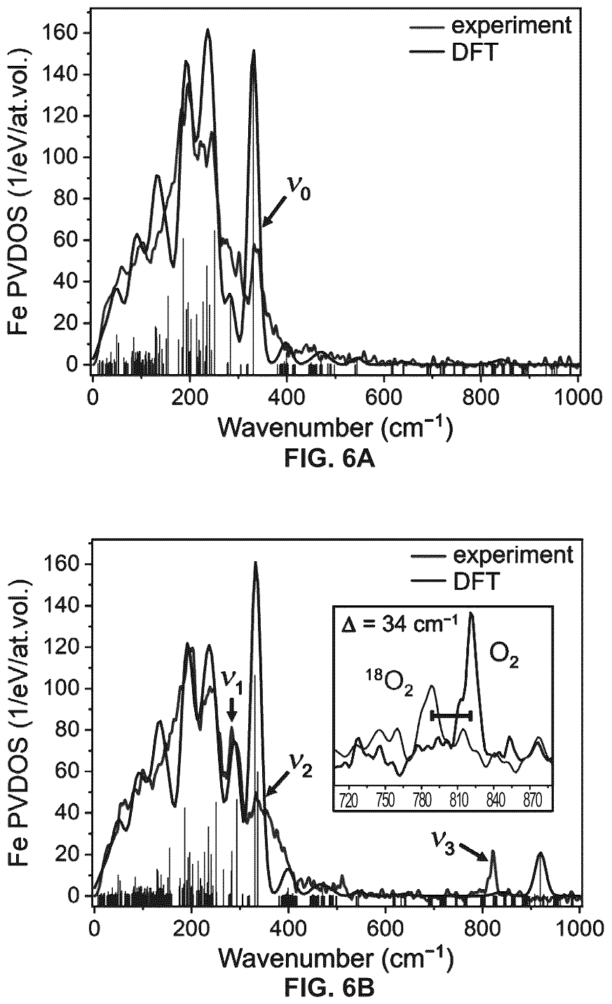

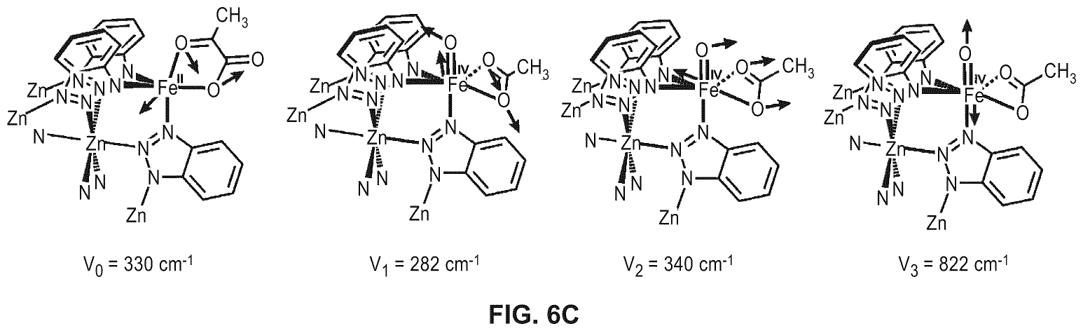

The iron partial vibrational density of states (PVDOS) distributions obtained from data collected at ˜100 K for desolvated 95% ⁵⁷Fe-enriched FeZn₄(prv)₄(btdd)₃ before and after ex situ dosing with 200 mbar of O₂ at 163 K is shown in FIG. 6A and FIG. 6B. Both experimental and DFT computed Fe PVDOS for FeZn₄(prv)₄(bta)₆ are presented in the figures.

A new peak at 822 cm⁻¹ for the O₂-dosed sample was assigned to an Fe(IV)=O vibration and is similar in magnitude to NRVS peaks reported for other nonheme Fe(IV)=O species in synthetic systems. In support of this assignment, when ¹⁸O₂ was employed for dosing, the vibration appeared instead at 788 cm⁻¹ (FIG. 6B inset). Consistent results were obtained from PVDOS distributions obtained for ⁵⁷Fe-enriched FeZn₄(moba)₄(btdd)₃ after dosing with 60 mbar O₂ or ¹⁸O₂ at 163 K, which feature peaks at 820 cm⁻¹ and 789 cm⁻¹, respectively, assigned to Fe═O and Fe=¹⁸O vibrations. The Fe(IV)=O peak was absent in the PVDOS distribution obtained for both frameworks after warming to 298 K.

Using the truncated cluster FeZn₄(prv)₄(bta)₆ as a model for FeZn₄(prv)₄(btdd)₃, DFT calculations were performed to simulate the NRVS iron PVDOS for the framework before and after O₂ dosing and is shown for comparison in FIGS. 6A and 6B. The intense stretch at 330 cm⁻¹ predicted for the model iron(II) framework corresponds to vibrations associated with bidentate pyruvate binding. Differences in the predicted and experimental intensities likely arise because the cluster model cannot fully describe the phonons of the framework lattice.

For the O₂-dosed sample, stretches at 282 and 340 cm⁻¹ are assigned as Fe—O vibrations resulting from K²-binding of the acetate ligand as shown in FIG. 6C. In contrast, Fe—O vibrations associated with K¹-binding of acetate are predicted to appear at higher wavenumbers (>400 cm⁻¹). The calculated Fe(IV)=O stretch is higher than the experimental stretching frequency (919 versus 822 cm⁻¹), likely because of a known systematic overestimation by DFT at these higher energies, which is less pronounced at lower energies.

Finally, simulated NRVS iron PVDOS were also generated for pristine and O₂-dosed FeZn₄(moba)₄(btdd)₃ from DFT calculations on the truncated S=2 cluster models Fe(moba)Zn₄(prv)₃(bta)₆ and Fe(O)(κ²—OPiv)Zn₄(prv)₃(bta)₆, and the results are in good agreement with the experimental results.

### Example 6

Reactivity studies were performed to demonstrate the capabilities of the frameworks. The reactivity of the FeₓZn₅₋ₓ(prv)₄(btdd)₃ framework with hydrocarbon substrates in the presence of O₂ was evaluated. A stoichiometric reaction of FeZn₄(moba)₄(btdd)₃ with O₂ and cyclohexane was first performed. Desolvated FeZn₄(prv)₄(btdd)₃ was suspended in cyclohexane and exposed to 1 bar of O₂ at 21° C. for 24 hours. Subsequently, CH₃CN-d₃ was added to extract the products, along with CH₂Br₂ as an internal standard. Analysis of the resulting supernatant using ¹H NMR spectroscopy and GC-MS revealed the formation of cyclohexanol (22% NMR yield with respect to the iron sites in the framework) with no detectable cyclohexanone.

Mössbauer spectroscopy analysis of the framework isolated following this reaction revealed only iron(II) species. The same stoichiometric reaction was also carried out using FeZn₄(moba)₄(btdd)₃ in the presence of O₂ (1 bar, 21° C.), and analysis of the resulting supernatant using ¹H NMR spectroscopy and GC-MS revealed the formation of only cyclohexanol with a 51% NMR yield with respect to the iron sites. A stoichiometric control reaction between cyclohexane and FeZn₄Cl₄(btdd)₃ in the presence of O₂ did not yield any hydrocarbon oxidation products.

In order to establish the direct role of the Fe(IV)=O species in C—H oxygenation, a stoichiometric reaction using a framework sample in which the Fe(IV)=O species was generated prior to the addition of substrate. A sample of FeZn₄(prv)₄(btdd)₃ was dosed with 200 mbar O₂ at 163 K, and after 2 hours, the sample headspace was evacuated, refilled with Ar and a mixture of cyclohexane and CD₂Cl₂ was added. The suspension was then warmed to 195 K and held for 2 hours and then allowed to warm to 294 K. Under these conditions, no cyclohexane oxidation products were detected via ¹H NMR spectroscopy.

When a similar reaction was performed using FeZn₄(moba)₄(btdd)₃, ¹H NMR spectroscopy and GC-MS analysis of the resulting supernatant revealed the formation of cyclohexanone (48% NMR yield). The ultimate formation of cyclohexanone in this case, in contrast to cyclohexanol formed in the reaction conducted at 21° C., was attributed to the lower reaction temperature and slower diffusion of cyclohexanol out of the framework pores, which is then further oxidized to cyclohexanone.

When the cyclohexane oxidation reaction with FeZn₄(prv)₄(btdd)₃ was repeated with the addition of 11 equivalents of pyruvic acid, cyclohexanone and cyclohexanol were obtained in a 2:1 ratio that produced a combined yield of 173% with respect to the total iron sites as illustrated in the process steps of FIG. 7. Powder x-ray diffraction analysis of the solid isolated from this reaction confirmed that the framework remains crystalline.

Significantly, this result suggests that FeZn₄(prv)₄(btdd)₃ can act as a catalyst in hydrocarbon oxidation reactions using the free α-keto acid as a co-substrate, presumably via a similar catalytic cycle as proposed for TauD. It was found that acetic acid byproduct is formed in this reaction in 288% yield with respect to the iron sites, which suggests 115% of pyruvic acid conversion to unidentified products. Such unproductive turnover at some of the iron sites is consistent with the relatively low yield in the stoichiometric reaction. A number of studies of synthetic and enzymatic systems have established that C—H bond activation by Fe(IV)=O species proceeds via H-atom abstraction, as evidenced by large primary kinetic isotope effects (KIEs). Consistent with these results, we determined an intermolecular competition KIE value of 29.8±1.0 from the reaction of FeZn₄(prv)₄(btdd)₃ with a mixture of cyclohexane and cyclohexane-d₁₂ under an atmosphere of O₂.

The reactivity of Fe_{1.8}Zn_{3.2}(prv)₄(btdd)₃ in the presence of O₂ was also evaluated using gaseous ethane as a substrate in a high-pressure batch reactor as illustrated in FIG. 8. Methane to methanol is shown in FIG. 9. Using a high-pressure mixture of ethane and dioxygen, ethanol and acetaldehyde were obtained in a 3:1 ratio with a high combined yield of 82%. This yield is much higher than that obtained in the stoichiometric cyclohexane oxidation, likely due to the high ethane concentration close to the iron sites under high pressure. Although there are enzymatic systems capable of oxidizing ethane to ethanol with O₂, our result is the first synthetic example of ethane oxidation via an unambiguously characterized S=2 Fe(IV)=O intermediate generated by 02.

It has been demonstrated that the iron(II) sites in the frameworks FeₓZn₅₋ₓ(prv)₄(btdd)₃ and FeZn₄(moba)₄(btdd)₃ activate O₂ at 100 K to form reactive high-spin Fe(IV)=O species. This reactivity is unprecedented in a synthetic system and highly reminiscent of O₂ activation in TauD. The Fe(IV)=O species were rigorously characterized using in situ DRIFTS, Mossbauer spectroscopy under zero and applied magnetic fields, Fe Kβ XES, and nuclear resonance vibrational spectroscopy.

From the description herein, it will be appreciated that the present disclosure encompasses multiple implementations of the technology which include, but are not limited to, the following:

A composition comprising FeₓZn₅₋ₓ(O₂CC(O)R)₄(btdd)₃, where R is an alkyl or aryl substituent.

The composition of any preceding or following implementation, wherein the alkyl or aryl substituent is a substituent selected from the group consisting of Et, n-Bu, t-Bu, 1-Pr, CF₃, Ph, Tol, C₆H₄CF₃ and C₆H₄OMe.

A composition comprising FeₓZn₅₋ₓ(prv)₄(btdd)₃ where (x=1.0 to 1.8).

A composition comprising FeZn₄(moba)₄(btdd)₃.

A method for chemoselective oxygenation of hydrocarbons, the method comprising: (a) providing a metal organic framework FeₓZn₅₋ₓ (02CC(O)R)₄(btdd)₃ where R is an alkyl or aryl substituent; (b) exposing the metal organic framework to oxygen gas and at least one hydrocarbon gas; and (c) collecting reaction products after a period of time.

The method of any preceding or following implementation, wherein the alkyl or aryl substituent of the metal organic framework is a substituent selected from the group consisting of Et, n-Bu, t-Bu, 1-Pr, CF₃, Ph, Tol, C₆H₄CF₃ and C₆H₄OMe.

The method of any preceding or following implementation, wherein the metal organic framework comprises FeₓZn₅₋ₓ(prv)₄(btdd)₃ where (x=1.0 to 1.8).

The method of any preceding or following implementation, wherein the metal organic framework comprises FeZn₄(moba)₄(btdd)₃.

A method for catalytic oxidation of cyclohexane to cyclohexanol and cyclohexanone, the method comprising: (a) providing a bed of metal organic framework materials of FeₓZn₅₋ₓ(prv)₄(btdd)₃; (b) adding a mixture of oxygen gas, cyclohexane, and α-ketoacid; (c) collecting oxidation products.

The method of any preceding or following implementation, wherein the α-ketoacid comprises pyruvic acid.

The method of any preceding or following implementation, wherein the metal organic framework material is selected from the group of Fe_{1.8}Zn_{3.2}(prv)₄(btdd)₃, Fe₁Zn₄(prv)₄(btdd)₃ and FeZn₄(moba)₄(btdd)₃.

A method for stoichiometric oxidation of ethane to ethanol and acetaldehyde, the method comprising: (a) providing a bed of a metal organic framework FeₓZn₅₋ₓ(O₂CC(O)R)₄(btdd)₃ where R is an alkyl or aryl substituent; (b) adding a mixture of oxygen gas and ethane over the bed; and (c) collecting ethanol and acetaldehyde oxidation products.

The method of any preceding or following implementation, wherein the alkyl or aryl substituent of the metal organic framework is a substituent selected from the group consisting of Et, n-Bu, t-Bu, 1-Pr, CF₃, Ph, Tol, C₆H₄CF₃ and C₆H₄OMe.

The method of any preceding or following implementation, wherein the metal organic framework comprises FeₓZn₅₋ₓ(O₂CC(O)R)₄(btdd)₃ where (x=1.0 to 1.8).

The method of any preceding or following implementation, wherein the metal organic framework comprises FeZn₄(moba)₄(btdd)₃.

The method of any preceding or following implementation, wherein the metal organic framework comprises Fe_{1.8}Zn_{3.2}(prv)₄(btdd)₃.

A method for oxidation of methane to methanol, the method comprising: (a) providing a bed of metal organic framework FeₓZn₅₋ₓ (O₂CC(O)R)₄(btdd)₃ where R is an alkyl or aryl substituent; (b) continuously flowing a mixture of oxygen gas and methane over the bed; and (c) collecting methanol oxidation products.

The method of any preceding or following implementation, wherein the alkyl or aryl substituent of the metal organic framework is a substituent selected from the group consisting of Et, n-Bu, t-Bu, 1-Pr, CF₃, Ph, Tol, C₆H₄CF₃ and C₆H₄OMe.

The method of any preceding or following implementation, wherein the metal organic framework comprises FeₓZn₅₋ₓ(O₂CC(O)R)₄(btdd)₃ where (x=1.0 to 1.8).

The method of any preceding or following implementation, wherein the metal organic framework comprises Fe_{1.8}Zn_{3.2}(prv)₄(btdd)₃.

The method of any preceding or following implementation, wherein the metal organic framework comprises FeZn₄(moba)₄(btdd)₃.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C,” within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment,” “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has . . . a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to 5°, less than or equal to 4°, less than or equal to 3°, less than or equal to 2°, less than or equal to 1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A composition comprising:
FeₓZn₅₋ₓ(O₂CC(O)R)₄(btdd)₃, where R is an alkyl or aryl substituent.

2. The composition of claim 1, wherein said alkyl or aryl substituent is a substituent selected from the group consisting of Et, n-Bu, t-Bu, 1-Pr, CF₃, Ph, Tol, C₆H₄CF₃ and C₆H₄OMe.

3. The composition of claim 1, comprising FeₓZn₅₋ₓ(prv)₄(btdd)₃ where (x=1.0 to 1.8).

4. The composition of claim 1, comprising FeZn₄(moba)₄(btdd)₃.

5. A method for chemoselective oxygenation of hydrocarbons, the method comprising:
(a) providing a metal organic framework FeₓZn₅₋ₓ(O₂CC(O)R)₄(btdd)₃ where R is an alkyl or aryl substituent;
(b) exposing the metal organic framework to oxygen gas and at least one hydrocarbon gas; and
(c) collecting reaction products after a period of time.

6. The method of claim 5, wherein said alkyl or aryl substituent of said metal organic framework is a substituent selected from the group consisting of Et, n-Bu, t-Bu, 1-Pr, CF₃, Ph, Tol, C₆H₄CF₃ and C₆H₄OMe.

7. The method of claim 5, wherein said metal organic framework comprises FeₓZn₅₋ₓ(prv)₄(btdd)₃ where (x=1.0 to 1.8).

8. The method of claim 5, wherein said metal organic framework comprises FeZn₄(moba)₄(btdd)₃.

9. A method for catalytic oxidation of cyclohexane to cyclohexanol and cyclohexanone, the method comprising:
(a) providing a bed of metal organic framework materials of FeₓZn₅₋ₓ(prv)₄(btdd)₃;
(b) adding a mixture of oxygen gas, cyclohexane, and α-ketoacid; and
(c) collecting oxidation products.

10. The method of claim 9, wherein said α-ketoacid comprises pyruvic acid.

11. The method of claim 9, wherein said metal organic framework material is selected from the group of Fe_{1.8}Zn_{3.2}(prv)₄(btdd)₃, Fe₁Zn₄(prv)₄(btdd)₃ and FeZn₄(moba)₄(btdd)₃.

12. A method for stoichiometric oxidation of ethane to ethanol and acetaldehyde, the method comprising:
(a) providing a bed of a metal organic framework FeₓZn₅₋ₓ(O₂CC(O)R)₄(btdd)₃ where R is an alkyl or aryl substituent;
(b) adding a mixture of oxygen gas and ethane over the bed; and
(c) collecting ethanol and acetaldehyde oxidation products.

13. The method of claim 12, wherein said alkyl or aryl substituent of said metal organic framework is a substituent selected from the group consisting of Et, n-Bu, t-Bu, 1-Pr, CF₃, Ph, Tol, C₆H₄CF₃ and C₆H₄OMe.

14. The method of claim 12, wherein said metal organic framework comprises FeₓZn₅₋ₓ(O₂CC(O)R)₄(btdd)₃ where (x=1.0 to 1.8).

15. The method of claim 12, wherein said metal organic framework comprises FeZn₄(moba)₄(btdd)₃.

16. The method of claim 12, wherein said metal organic framework comprises Fe_{1.8}Zn_{3.2}(prv)₄(btdd)₃.

17. A method for oxidation of methane to methanol, the method comprising:
(a) providing a bed of metal organic framework FeₓZn₅₋ₓ(O₂CC(O)R)₄(btdd)₃ where R is an alkyl or aryl substituent;
(b) continuously flowing a mixture of oxygen gas and methane over the bed; and
(c) collecting methanol oxidation products.

18. The method of claim 17, wherein said alkyl or aryl substituent of said metal organic framework is a substituent selected from the group consisting of Et, n-Bu, t-Bu, 1-Pr, CF₃, Ph, Tol, C₆H₄CF₃ and C₆H₄OMe.

19. The method of claim 17, wherein said metal organic framework comprises FeₓZn₅₋ₓ(O₂CC(O)R)₄(btdd)₃ where (x=1.0 to 1.8).

20. The method of claim 17, wherein said metal organic framework comprises Fe_{1.8}Zn_{3.2}(prv)₄(btdd)₃.

21. The method of claim 17, wherein said metal organic framework comprises FeZn₄(moba)₄(btdd)₃.
